(19) United States
(12) Patent Application Publication
(10) Pub. No.: US 2007/0178477 A1
(43) Pub. Date: Aug. 2, 2007

(54) Nanotube sensor devices for DNA detection

(75) Inventors: Charles S. Joiner JR., Cupertino, CA (US); Jean-Christophe P. Gabriel, Pinole, CA (US); George Gruner, Los Angeles, CA (US); Alexander Star, Pittsburgh, PA (US)

Correspondence Address: O'MELVENY & MYERS LLP, 610 NEWPORT CENTER DRIVE, 17TH FLOOR, NEWPORT BEACH, CA, 92660, US

(73) Assignee: Nanomix, Inc., Emeryville, CA (US)

(21) Appl. No.: 11/212,026

(22) Filed: Aug. 24, 2005

Related U.S. Application Data
(63) Continuation-in-part of application No. 10/345,783, filed on Jan. 16, 2003.
(63) Continuation-in-part of application No. 10/704,066, filed on Nov. 7, 2003.
(60) Provisional application No. 60/604,293, filed on Aug. 24, 2004.
(60) Provisional application No. 60/629,604, filed on Nov. 19, 2004.
(60) Provisional application No. 60/349,670, filed on Jan. 16, 2002.
(60) Provisional application No. 60/424,892, filed on Nov. 8, 2002.

(51) Int. Cl. C12Q 1/68; C12M 3/00

(52) U.S. Cl. 435/6; 435/287.2; 977/924

(57) ABSTRACT

A nanotube device is configured as an electronic sensor for a target DNA sequence. A film of nanotubes is deposited over electrodes on a substrate. A solution of single-strand DNA is prepared so as to be complementary to a target DNA sequence. The DNA solution is deposited over the electrodes, dried, and removed from the substrate except in a region between the electrodes. The resulting structure includes strands of the desired DNA sequence in direct contact with nanotubes between opposing electrodes, to form a sensor that is electrically responsive to the presence of target DNA strands. Alternative assay embodiments are described which employ linker groups to attach ssDNA probes to the nanotube sensor device.

### CROSS-REFERENCE TO RELATED APPLICATION

This application claims priority pursuant to 35 U.S.C. §119(e) to U.S. Provisional Application No. 60/604,293, filed Aug. 24, 2004, and to U.S. Provisional Application No. 60/629,604, filed Nov. 19, 2004, each of which applications is specifically incorporated herein, in its entirety, by reference.

This application also claims priority as a continuation-in-part of U.S. patent application Ser. No. 10/345,783 filed Jan. 16, 2003, entitled “Electronic Sensing of Biological and Chemical Agents Using Functionalized Nanostructures” (now published as 2003-0134433), which claims priority to U.S. Provisional Patent Application No. 60/349,670 filed Jan. 16, 2002; each of which applications is specifically incorporated herein, in its entirety, by reference.

This application also claims priority as a continuation-in-part of U.S. patent application Ser. No. 10/704,066 filed Nov. 7, 2003 entitled “Nanotube-Based Electronic Detection Of Biomolecules” (published as US 2004-0132070 on Jul. 8, 2004), which claims priority to U.S. Provisional Patent Application No. 60/424,892 filed Nov. 8, 2002, each of which applications is specifically incorporated herein, in its entirety, by reference.

### BACKGROUND OF THE INVENTION

1. Field of the Invention

The present invention relates to sensors for specific DNA sequences, using nanotubes as electronic transducers of DNA hybridization.

2. Description of Related Art

Because base sequences in polynucleotides encode genetic information, the ability to read these sequences has contributed to many advances in biotechnology. This work has identified many important sequences that are linked to medical conditions. For example, the BRCA gene is usually present in women who suffer from breast cancer. To take advantages of these linkages in medical testing, various techniques have been developed to scan tissue samples for the occurrence of specific important sequences. These techniques have shortcomings that make them expensive, slow, and complex, so that they are unlikely to be useful for routine medical testing.

These techniques universally rely on the tendency of polynucleotides to hybridize. A strand of single-strand DNA (ssDNA) in solution readily combines with a complementary strand (cDNA) that contains an opposite base to pair with each base in the ssDNA. The result of this combination is double-stranded DNA (dsDNA), which can be processed and separated from ssDNA. Thus, to scan for a particular target sequence, an experimenter provides the appropriate cDNA as a probe sequence. If the target sequence is present in a sample, the target ssDNA will hybridize with the probe ssDNA to produce dsDNA, and this hybridization can be detected in some way.

A first shortcoming arises because many methods of detecting this hybridization involve modification of the sample ssDNA before hybridization. Often, a fluorescent molecule is attached to the ssDNA. This molecule, known as a label, causes the ssDNA to be detected by optical instruments such as microscopes and spectrometers. Labeling is used to detect sample DNA after a hybridization step. If the target sequence is present in a labeled sample, the labeled ssDNA will be incorporated in labeled dsDNA, and the dsDNA will thus be detectable with optical instruments. Although the use of optical detection makes this approach convenient, the chemical reaction by which the DNA Is labeled is expensive and time-consuming. A detection method which did not require labeling would significantly increase the usefulness of DNA scanning for routine medical tests.

A second problem results from the low sensitivity of traditional detection methods. Although some of these methods are sensitive to low concentrations of DNA, they require large absolute numbers of DNA molecules. In a medical application, only a few cells are usually available, and consequently only a few DNA molecules of the target sequence will be present in a sample. This problem has been ameliorated by the use of the polymerase chain reaction (PCR), which can amplify the quantity of target DNA a million-fold. Like labeling, PCR is a complex chemical reaction, which makes tests expensive and slow.

Thus, there is a clear need for a sensitive, fast, technique for detecting specific target DNA sequences. Such a technique should operate without the use of PCR or labeling.

### SUMMARY OF THE INVENTION

The invention provides an electronic sensor device with which to detect specific target sequences of polynucleotides. The sensor comprises nanostructured elements, (for example single and/or multiwalled carbon nanotubes and/or interconnecting networks comprising such nanotubes) which interact with polynucleotides so as to act as sensing elements. In the particular examples described in detail, the nanostructured elements comprise carbon nanotubes, and more particularly, randomly oriented networks of carbon nanotubes. In these examples, the nanotubes are modified before sensing by the adsorption of ssDNA probe sequences. No labeling of the DNA is required. Further, the invention provides a method for using the sensor device.

As used herein, a “nanostructure” is any object which has at least one dimension smaller than 100 nm and comprises at least one sheet of crystalline material with graphite-like chemical bonds. Examples include, but are not limited to, single-walled nanotubes, double-walled nanotubes, multi-walled nanotubes, and “onions.” Chemical constituents” of the crystalline material include, but are not limited to, carbon, boron nitride, molybdenum disulfide, and tungsten disulfide.

For simplicity, the nanostructures included in the examples described in detail may be referred to as “nanotubes”, and exemplary embodiments preferably include one or more carbon nanotubes, and more preferably one or more single-walled carbon nanotubes. It is noted that alternative embodiments may include alternative nanostructures in nanostructured sensor elements without departing from the spirit of the invention.

A “nanotube network”, as used herein, is a film of nanotubes disposed on a substrate in a defined area. A film of nanotubes comprises at least one nanotube disposed on a substrate in such a way that the nanotube is substantially parallel to the substrate. The film may comprise many nanotubes oriented parallel to each other. Alternatively, the film may comprise many nanotubes oriented randomly. The film may comprise few nanotubes in a selected area of substrate, or the film may comprise many nanotubes in a selected area of substrate. The number of nanotubes in an area of substrate is referred to as the density of a network. Preferably, the film comprises many nanotubes oriented randomly, with the density high enough that electric current may pass through the network from one side of the defined area to the other side, such as via nanotube-to-nanotube contact points.

Substrates are flat objects that typically include an electrically insulating surface. Substrates have a chemical composition, of which examples include, but are not limited to, silicon oxide, silicon nitride, aluminum oxide, polyimide, and polycarbonate. In a number of examples described herein, the substrate includes one or more layers, films or coatings comprising such materials as silicon oxide, SIO₂, Si₃N₄, and the like, upon the surface of a silicon wafer or chip.

Nanotube networks may be made by such methods as chemical vapor deposition (CVD) with traditional lithography, by solvent suspension deposition, vacuum deposition, and the like. See for example, U.S. patent application Ser. No. 10/177,929 (corresponding to WO2004-040,671); U.S. patent application Ser. No. 10/280,265; U.S. patent application Ser. No. 10/846,072; and L. Hu et al., Percolation in Transparent and Conducting Carbon Nanotube Networks, Nano Letters (2004), 4, 12, 2513-17, each of which applications and publication is incorporated herein by reference.

Properties of the nanostructure elements (e.g., nanotube network) may by measured using contacts. A contact includes a conducting element disposed such that the conducting element is in electrical communication with the nanostructure element, such as a nanotube network. For example, contacts may be disposed directly on a substrate surface, or alternatively may by disposed over a nanotube network. Electric current flowing in the nanotube network may be measured by employing at least two contacts that are placed within the defined area of the nanotube network, such that each contact is in electrical communication with the network.

In some embodiments of the invention, an additional conducting element, referred to as a gate or counter electrode, is provided such that it is not in electrical communication with the nanostructured element (such as at least one nanotube), but such that there is an electrical capacitance between the gate electrode and the nanostructured element. In a preferred embodiment, the gate electrode is a conducting plane within the substrate beneath the silicon oxide. Examples of such nanotube electronic devices are provided, among other places, in patent application Ser. No. 10/656,898, filed Sep. 5, 2003 and Ser. No. 10/704,066, filed Nov. 7, 2003 (published as US 2004-0132,070), both of which are incorporated herein, in their entirety, by reference. Resistance, impedance, transconductance or other properties of the nanotubes may be measured under the influence of a selected or variable gate voltage.

In another preferred embodiment, the gate electrode is a conducting element in contact with a conducting liquid, said liquid being in contact with the nanotube network. Examples of this embodiment are provided, among other places, in Bradley et al., Phys. Rev. Lett. 91, 218301 (2003), which is incorporated herein, in its entirety, by reference.

In other examples, a voltage may be applied to one or more contacts to induce an electrical field in a nanotube network relative to a counter electrode or gate electrode, and the capacitance of the network may be measured. Conveniently, the source (and/or drain) and gate electrodes of a transistor having a nanostructured channel (e.g., nanotube network) may be employed using suitable circuitry to measure the capacitance of the channel relative to the gate, as an alternative or additional sensor signal to measurements of one or more channel transconductance properties. Alternative embodiments configured to optimize measurements of capacitance or other properties are possible without departing from the spirit of the invention.

The conducting elements provide for connecting to an electrical circuit for observing an electrical property of the nanotube sensor. Any suitable electrical property may provide the basis for sensor sensitivity, for example, electrical resistance, electrical conductance, current, voltage, capacitance, transistor on current, transistor off current, or transistor threshold voltage. Those skilled in the art will appreciate that other electrical properties may also readily be observed and measured. Accordingly, this list is not meant to be restrictive of the types of device properties that can be measured.

In a preferred embodiment, a nanotube sensor device includes a transistor. A transistor has a maximum conductance, which is the greatest conductance measured with the gate voltage in a range, and a minimum conductance, which is the least conductance measured with the gate voltage in a range. A transistor has an on-off ratio, which is the ratio between the maximum conductance and the minimum conductance. To make a sensitive chemical sensors, a nanotube transistor has an on-off ratio preferably greater than 1.2, more preferably greater than 2, and most preferably greater than 10.

For example, FIG. 1 shows an exemplary conductance curve as a function of gate voltage between +10 V and −10 V for a nanotube electronic device. Relatively high conductance in the “on” curve portion 101 occurs at gate voltages less than about −5 V; relatively low conductance in the “off” curve portion 102 occurs at gate voltages greater than about 0 V. For this device, the on/off ratio is about 100.

As used herein, “DNA” means polynucleotides. Examples of polynucleotides include, but are not limited to, deoxyribonucleic acid, ribonucleic acid, messenger ribonucleic acid, transfer ribonucleic acid, and peptide nucleic acid. The defining characteristics of polynucleotides are a chain of nucleic acids and a sequence of bases, each base chemically bonded to a nucleic acid and each base capable of pairing with an appropriate base on a matching sequence. Those skilled in the art will appreciate that other variations of polynucleotides may be produced which share these defining characteristics. Accordingly, a “single-strand strand DNA”, referred to hereafter as “ssDNA”, may be a single strand of deoxyribonucleic acid, ribonucleic acid, or any other polynucleotide as described above. A “double-strand DNA”, referred to hereafter as “dsDNA”, may be a double strand of any polynucleotide described above. “Complimentary DNA”, referred to hereafter as “cDNA”, may be any strand of a polynucleotide described above which is a single-strand sequence complimentary to an already referenced single-strand sequence.

In certain embodiments, the invention provides a nanotube sensor device comprising a nanotube network, one or more contacts, and ssDNA contacting the nanotubes. Multiple methods are available for preparing the ssDNA contacting the nanotubes. In one embodiment, ssDNA in solution is mixed with nanotubes in suspension, as described in by Zheng, M. et al. in Nature Materials 2003, 2, 338-342. The resulting solution contains nanotubes around which are wrapped ssDNA strands. The solution is cast onto a substrate, so that ssDNA-wrapped nanotubes are disposed on the substrate. After the disposal of the nanotubes, contacts are made using standard techniques of lithography and metal deposition. In a preferred embodiment, a nanotube network is disposed on a substrate and contacts are made. The resulting electronic device is exposed to a solution containing ssDNA. When the solution is removed, it is found that ssDNA has coated the nanotube network, without coating the substrate.

In certain embodiments, the invention provides devices in which ssDNA contacts the nanotubes directly, without the use of an intervening linker molecule. Further, the ssDNA contacts the nanotubes but does not contact the substrate in areas which are not contacted by nanotubes.

The ssDNA in a particular sensor device may be selected to be cDNA for a particular target sequence. The target sequence is the sequence of bases that the sensor device is intended to detect. The cDNA for the target sequence is known as the probe sequence. Once a target sequence is specified, a quantity of DNA with the probe sequence must be obtained. A variety of techniques are known for synthesizing DNA with specified sequences and for synthesizing DNA complementary to a given sequence. Those skilled in the art will have knowledge of these techniques. Further, appropriate cDNA or other polynucleotide to make a probe specific to a desired target sequence can generally be obtained from known commercial suppliers serving the biotechnology industry.

A sensor device may be used by exposing the nanotube network to a solution containing sample ssDNA. The network should be exposed to the solution for a period of time long enough for hybridization to occur. This period of time depends on the concentration of the sample DNA, the quantity of the solution, the temperature of the room, the pH of the solution, and other variables. Those skilled in the art are familiar with the effect of these variables on DNA hybridization and are capable of choosing an appropriate period of time, solution composition, temperature and other conditions of hybridization without undue experimentation.

Multiple methods of using the sensor devices are disclosed.

In one embodiment, the sensor device is first measured by varying a gate voltage applied by a conducting plane beneath the insulator of the substrate. Then the network is exposed to a solution containing sample ssDNA for the period of time disclosed above. Next, the solution is removed, and a period of time is allowed to lapse sufficient for the substrate to become substantially dry. This period of time may be made briefer by taking actions which speed the drying process. For example, dry air may be blown over the substrate. After the substrate is dry, the sensor device is measured again by varying the gate voltage. The resulting measurement is compared to the first measurement to see if dsDNA is present.

In another embodiment, the network is exposed to pure water to obtain a baseline. The sensor device is first measured by varying a gate voltage applied by a conducting plane beneath the insulator of the substrate. Then the network is exposed to a solution of sample DNA in pure water. If the sample DNA contains target DNA, hybridization may occur over time, and the resulting measurement of the sensor device changes in comparison to the first measurement.

In yet another embodiment, the network is exposed to pure water to obtain a baseline. The sensor device is first measured by varying a gate voltage applied by a conducting plane beneath the insulator of the substrate. Then the network is exposed to a solution of sample DNA in a buffer compounded (in terms of temperature, pH, dissolved species, and the like) to promote hybridization. Following a period of time for hybridization, the network may be washed to remove unhybridized DNA and other material. Following washing, the network is again exposed to pure water, and the measurement is repeated. If the sample DNA contains target DNA, hybridization of this DNA will result measurable changes in sensor device characteristics in comparison to the first measurement.

In yet another embodiment, the baseline measurement is performed in the same buffer as is used for hybridization. Then the network is exposed to a solution of sample DNA in the hybridization buffer. Following a period of time for hybridization, the measurement is repeated. If the sample DNA contains target DNA, hybridization of this DNA will result measurable changes in sensor device characteristics in comparison to the first measurement.

In another embodiment, the network is exposed to a conducting liquid. Preferably, the conducting liquid is a buffer appropriate for physiological fluids; most preferably, the conducting liquid is phosphate buffer solution (PBS). The sensor device is first measured by varying a gate voltage applied by a conducting element in contact with the conducting liquid. Then the network is exposed to a solution of sample DNA in a similar conducting liquid. While the network is exposed, the sensor device is measured by varying the gate voltage. If the sample DNA contains target DNA, hybridization occurs over time, and the resulting measurement of the sensor device changes in comparison to the first measurement.

In yet other embodiments, an electronic sensor system comprises sensor platform having a substrate, one or more electrodes, a nanostructured element disposed adjacent the substrate in electrical communication with at least one of the electrodes; and electronic measurement circuitry connected to the electrodes and configured to measure one or more electrical properties of the sensor platform. The sensor system includes at least one detector probe operatively associated with the sensor platform, the probe including (a) a linker group disposed in association with the sensor platform, the linker being connected to one or more of the following: the nanostructured element, the substrate, and the electrode; (b) a detector biomolecule having a binding affinity to an analyte polynucleotide; and (c) a bonding connection between the linker group and the detector biomolecule. The detector biomolecule may include species having a selective affinity for a polynucleotide, such as a complementary polynucleotide, a transcription factor and/or a transcription promoter, or synthetic versions or analogs of these. In preferred embodiments, the detector biomolecule comprises a detector polynucleotide having at least one nucleotide sequence which is at least partially complementary to a nucleotide target sequence of the analyte polynucleotide. In the examples described in detail, the sensor system measures a property in influenced by engagement of the probe with an analyte polynucleotide by at least partial hybridization of the target sequence.”

It should be noted that, with respect to all the described sensor embodiments, that the occurrence, speed and specificity of polynucleotide hybridization depends on various conditions. In each of these hybridization schemes, the binding energy of the dsDNA can be challenged through stringency techniques. This can be done through temperature increases or buffer changes, for example sodium hydroxide.

Additional stringency controls may include various ionic constituents of the hybridization medium, such as sodium or magnesium ions. Alternatively or additionally, a voltage may be applied to elements of the sensor (e.g., a nanotube network) before, during and/or after hybridization to influence polynucleotide behavior. For example, a polynucleotide such as cDNA has a phosphate-based backbone which typically is ionized in the hybridization medium so as to carry a localized negative charge. Selectively charged sensor elements may be used to provide an attractive or repulsive stringency factor, for example, to destabilize a SNP-mismatched probe hybrid relative to a corresponding fully-matched probe hybrid (e.g., during incubation or during a rinse process).

Through variations in stringency, it is possible to differentiate binding of strands with complete or incomplete complementary base pairs. Changes in electrical properties of the nanotubes in response to the stringency process allow discrimination of single base mismatches (SNP), among other things. One of ordinary skill in the art will be able to vary the hybridization conditions so as to tune the operation of certain embodiments of the sensors of the invention to obtain a selected degree of sensitivity to complete and less-than-complete hybridization of the target sequence.

For example, in an assay to discriminate between a DNA sample which is homozygous for a particular allele, on the one hand, and an otherwise comparable sample which is heterozygous for this allele, the stringency of the hybridization conditions may be adjusted (e.g. by variation in temperature) so as to produce a distinctly different device measurement response between the homozygous and heterozygous samples.

In the case of each of the sensor embodiments having aspects of the invention, these sensors may be constructed in arrays, e.g. arrays of transistor sensors functionalized for a plurality of different target DNA fragments. See application Ser. No. 10/388,701 entitled “Modification Of Selectivity For Sensing For Nanostructure Device Arrays” (published as US 2003-0175,161), incorporated by reference above.

A more complete understanding of the nanotube sensor devices will be afforded to those skilled in the art, as well as a realization of additional advantages and objects thereof, by a consideration of the following detailed description of the preferred embodiment. Reference will be made to the appended sheets of drawings which will first be described briefly.

### BRIEF DESCRIPTION OF THE DRAWINGS

FIG. 1 is a schematic diagram showing an exemplary conductance curve for a nanotube transistor device.

FIG. 2 is a schematic diagram showing an exemplary design for a nanotube sensor using a random network of nanotubes.

FIG. 3 is a schematic cross-sectional diagram showing the exemplary nanotube sensor of FIG. 2.

FIG. 4 is a flow chart showing exemplary steps of a method for making a nanoelectronic sensor according to the invention, and as described in Example A.

FIG. 5 is a flow chart showing exemplary steps of a method for sensing an polynucleotide according to the invention.

FIG. 6 is a chart showing conductance as a function of gate voltage for a nanotube electronic device in three circumstances, as described further in the detailed description of the preferred embodiment.

FIG. 7A shows the device characteristics of the sensor of Example B after functionalization with the pyrene-DNA conjugate and treatment with cDNA.

FIG. 7B Shows the device characteristics of the sensor of Example B after functionalization with the pyrene-DNA conjugate, treatment with SNP-DNA, and subsequent treatment with cDNA.

FIG. 8A shows an exemplary DNA assay embodiment according to certain aspects of the invention, employing a detector probe linked to the sensor.

FIGS. 8B-F shows an alternative DNA assay embodiment according to certain aspects of the invention, employing electroactive incalators.

FIGS. 8A-D shows an alternative DNA assay embodiment according to certain aspects of the invention, employing amplifier groups.

FIGS. 9A-B shows an alternative DNA assay embodiment according to certain aspects of the invention, employing antibody-antigen binding to link the detector probe to the sensor.

FIGS. 10A-D shows two alternative sensor architectures according to certain aspects of the invention, in which the detector probe is linked to nanostructures such as nanotubes.

FIGS. 11A-C shows two alternative sensor architectures according to certain aspects of the invention, in which the detector probe is linked to the sensor substrate.

FIGS. 12A-B shows two alternative sensor architectures according to certain aspects of the invention, in which the detector probe is linked to the sensor electrodes.

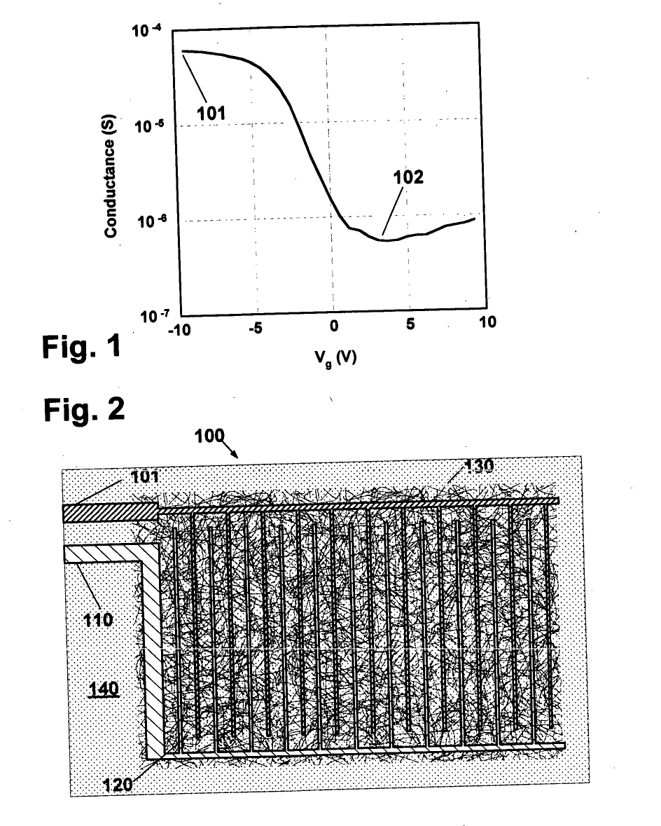

### DETAILED DESCRIPTION OF THE PREFERRED EMBODIMENT

In certain embodiments, the present invention provides a nanotube sensor device that detects a target DNA sequence. The device requires no labeling of the target DNA and responds electronically to the presence of the target DNA. In the detailed description that follows, like element numerals are used to indicate like elements appearing in one or more of the figures.

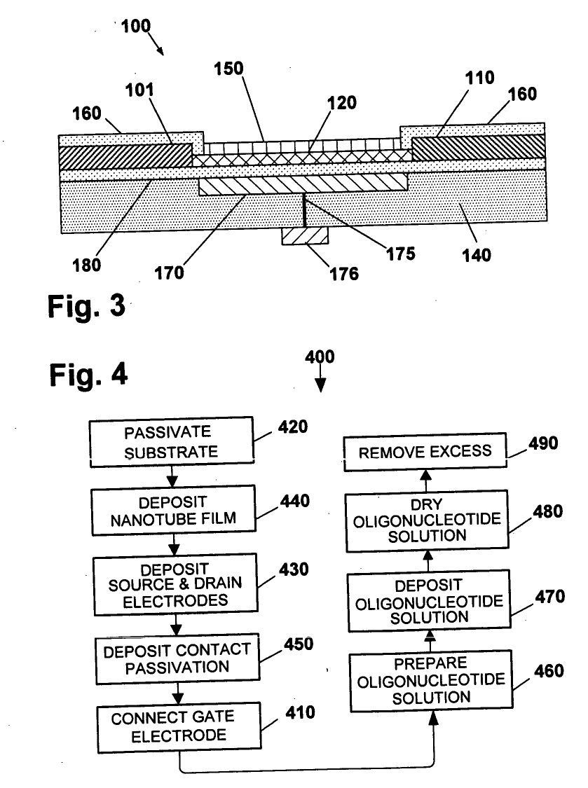

Referring to FIGS. 2 and 3, a nanotube DNA sensor 100 according to the invention may comprise a suitable substrate 140, for example, a degenerately doped silicon wafer. Other substrates may include, for example, other semiconductors, or insulating substrates such as ceramics or polymers. Substrate 140 may be passivated with a silicon oxide film 180, as known in the art.

Optionally, a gate electrode 170 may be formed in a lower layer of the substrate, and connected to a contact 176 via any suitable conductor 175. Alternatively, the substrate may comprise a conducting base material, such as doped silicon, covered by an insulating layer, such as S102, in which the conducting base material is connected to circuitry to serve as a gate or counter electrode.

In the example shown, a network of randomly oriented nanotubes 120 is disposed over a silicon substrate 140, and the device includes a pair of contacts 101, 110 having interdigitated portions disposed over network 120, the network providing a conducting channel between the contact pair. The substrate 140 outside of the generally rectangular area 130 should be substantially free of the nanotube network 120.

Alternative embodiments may comprise a single or a plurality of nanotubes disposed adjacent a substrate, in which the nanotubes are in electrical contact with one or more contacts. In some embodiments, most or all of the nanotubes may span to electrically conduct between a pair of adjacent contacts.

In the randomly oriented interconnecting nanotube network 120 of the example shown, however, it is not necessary that all or a majority of the nanotube be in electrical contact with one or more electrodes. Inter-nanotube contacts may serve to provide a conductive path, permitting current or charge transmission through the network.

In the example shown, the contacts 101, 110 are deposited over network 120. Alternatively, contacts may be deposited upon substrate 140, and network 120 formed upon the contacts.

One or more of contacts 101, 110 may be provided, and may optionally have a covering passivation layer 180, as known in the art. For example, contacts 101, 110 may comprise one or more metal layers, such as titanium and gold.

Contacts 101, 110 may comprise a plurality of interdigitated portions disposed over a generally rectangular region 130. The interdigitated configuration advantageously increases the surface area of the contacts that can be exposed to a nanotube film between the contacts. Other configurations of contacts may also be suitable, for example, parallel labyrinths of any desired shape, or any other configuration providing a sensor region between opposing contacts. The rectangular shape of region 130 is merely exemplary, and this region may comprise any desired shape. Contacts 101, 110 may be configured as source and drain electrodes for a field-effect transistor device or merely serve as connections to a resistive or capacitive sensor. In certain embodiments, a single contact (e.g., 101) may be employed to induce an electrical field or capacitance of the network 120 relative to gate electrode 170 or other counter electrode, so as to provide a sensor signal.

Contacts 101, 110 and portions of the substrate 140 that are not between the contacts may be protected by a barrier material 160. For example, an epoxy resin, or any other suitable polymer or resin material, may be deposited to form a barrier 160, and removed, such as by etching, from a region between the opposing contacts 101, 110.

A plurality of single-strand DNA molecules 150 may be disposed over the nanotube film using any suitable method, for example as described herein below. The DNA molecules may be attached directly to nanotubes in the nanotube film 120, or may rest on the substrate 140 near nanotubes in the film. In the alternative, DNA molecules may be disposed over a material interposed between the nanotube film and the DNA. The DNA should, however, be disposed sufficiently close to the nanotube film so that a reaction between the ssDNA and complementary ssDNA strands influences a measured electrical property of sensor 100.

In one exemplary embodiment of the invention (see Example A), the ssDNA contacts the nanotubes directly, without the use of an intervening linker molecule. Further, the ssDNA contacts the nanotubes but does not contact the substrate in areas which are not contacted by nanotubes. The ssDNA molecule 150 may be removed from substrate 140 except from over the nanotube film 120.

The ssDNA in a particular sensor device is selected to be cDNA for a particular target sequence. The target sequence is the sequence of bases that the sensor device is intended to detect. The cDNA for the target sequence is known as the probe sequence. Once a target sequence is specified, a quantity of DNA with the probe sequence must be obtained. A variety of techniques are known for synthesizing DNA with specified sequences and for synthesizing DNA complementary to a given sequence. Those skilled in the art will have knowledge of these techniques. Further, cDNA may often be obtained from commercial sources.

It should be appreciated that a plurality of nanotube sensors like sensor 100 may be formed in parallel on a single substrate, and later separated. Separated devices may be mounted in chip carriers as known in the art, and integrated with conventional electronics to provide useful sensing instrumentation that should be capable of sensing a targeted polynucleotide. Multiple sensors sensitive to different sequences may be combined in an electronic device to detect a variety of different polynucleotide sequences at once. One of ordinary skill may construct suitable electronics for a sensing instrument, using the disclosure herein.

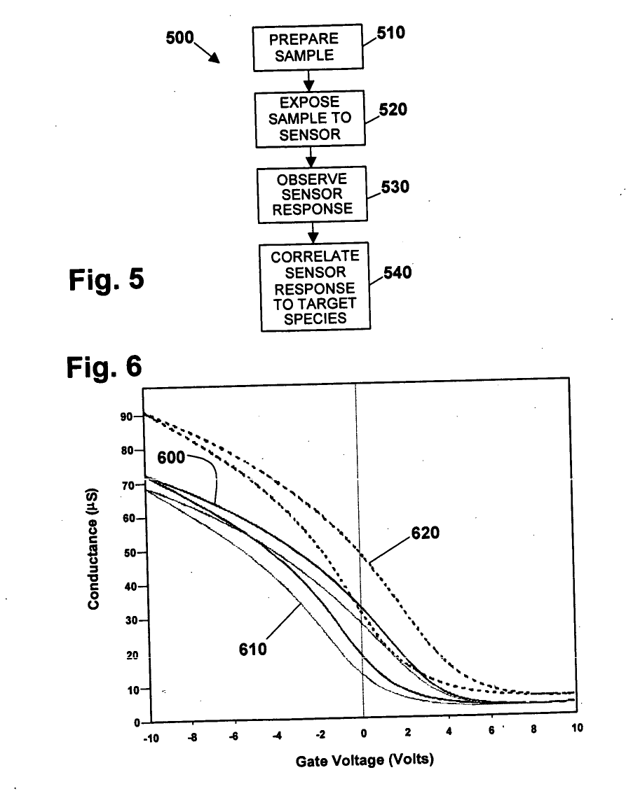

FIG. 4 shows exemplary steps in a method 400 for making a nanoelectronic sensor for particular DNA sequences. Steps 410 through 490 may be performed in any operative order.

At step 410, a gate electrode may be formed on a substrate, for example a passivated silicon or other semiconducting substrate, or on a semiconducting substrate such as a ceramic or polymer material. The electrode may comprise a metal or other conducting material, and may be formed using photolithography and lift-off as known in the art, or any other suitable method. In certain embodiments, the gate electrode comprises bulk silicon substrate wafer material, connected to suitable circuitry.

At step 420, the substrate (and embedded gate electrode, if included) may be coated with a passivation or insulating layer, such as a silicon oxide layer, as known in the art.

At step 440, one or more nanotubes is placed in the substrate in electrical communication with each of the opposing contacts. For example, the substrate 140 may be coated with carbon nanotubes in a random network, as described in the earlier-referenced U.S. patent application Ser. No. 10/177,929. In the alternative, other methods as known in the art for forming nanotubes between contacts may be used. The resulting nanotubes may be oriented in a specified fashion, or randomly oriented. If randomly oriented, the nanotubes should provide a network of connected nanotubes that connects the opposing contacts via at least one pathway. Nanotubes should be removed from the substrate in areas other than between the opposing documents, using any suitable method, such as plasma etching.

At step 430, a pair of opposing contacts, such as source and drain electrodes, may be formed on the substrate. The contacts may be above the nanotubes, or may be between the nanotubes and the substrate. For example, titanium contacts may be formed and covered with a gold layer using photolithography and lift-off to form opposing contacts. The contacts may comprise a plurality of interdigitated portions disposed over an intermediate region of any desired shape.

At step 450, an optional layer of barrier material may be deposited over the contacts. Various polymers and resins are known in the art, and may comprise a suitable barrier. In an embodiment of the invention, an epoxy coating may be used. The barrier may be applied only in certain areas of the substrate, or applied over the entire substrate and removed from operative areas of the sensor such as between the contacts. The barrier may provide for electrical insulation, preventing short-circuiting of the sensor when in contact with an conductive fluid, or otherwise protecting the sensor from exposure to the environment. The barrier may also be helpful in controlling the deposition of other materials, including but not limited to nanotubes and DNA molecules. Any number of barrier layers may be used.

At step 460, a solution of oligonucleotide (ssDNA) may be prepared. The desired ssDNA (“probe sequence”) may be obtained from a commercial source or synthesized as known in the art. A water or organic solution of the probe sequence may be prepared at a suitable concentration. For example, a solution of 10⁻⁴ M concentration may be prepared by dissolving 100,000 p mole of the oligonucleotide in 1000 μL of pure (18 MΩ). Other solvents compatible with ssDNA may be used. Prior to depositing the ssDNA, the electrical properties of the sensor device may optionally be noted as a baseline.

At step 470, the oligonucleotide solution may be applied over the active region 130 of the sensor device. For example, a drop of DNA solution may be placed on the chip over region 130. Then, the solution may be dried to evaporate the carrier and leave the ssDNA behind intact. For example, the may be placed in a humidified chamber at room temperature until dry. Then the chip may be removed from the chamber, rinsed with 18 MΩ water and blown dry with dry nitrogen. At step 490, excess ssDNA may be removed. This may occur by rinsing and blowing, as just described. More aggressive methods, e.g., etching, may be used if excess DNA is bonded to other areas of the substrate. In the alternative, excess DNA may be left in place if doing so does not disrupt sensor operation.

The electrical properties of the sensor device may be again observed and compared to the baseline properties. To the extent ssDNA has been successfully deposited, a change in the electrical properties should be observable. Properties that may be observed may include, for example, sensor gate voltage, conductance, resistance, or any combination, curve or hysteresis involving these or other electrical properties.

FIG. 5 shows exemplary steps of a method 500 for using a sensor device according to the invention. Essentially, a sensor is used by exposing the nanotube network to a solution containing sample ssDNA, and observing changes in the electrical properties of the sensor. At step 510, the sample is prepared as known in the art. For example, DNA may be extracted from a patient's cells by dissolution. Double-stranded DNA should be reduced to ssDNA using a method as known in the art. If a sufficiently large sample of DNA is available, if may be possible to avoid use of a PCR method to increase DNA concentration. Since the sensor of the present invention may operate using an extremely small sample volume (e.g., less than 100 μL), use of PCR may in some instances be avoided.

At step 520, the sensor is exposed to the sample solution. The sensor should be left in the solution for a period of time long enough for hybridization to occur between at least one ssDNA molecule on the nanotube network and a complementary ssDNA molecule in solution. This period of time depends on the concentration of the sample DNA, the quantity of the solution, the temperature of the room, the pH of the solution, and other variables. Those skilled in the art are familiar with the effect of these variables on DNA hybridization and are capable of choosing an appropriate period of time.

At step 530, an electrical response of the sensor is observed. Various different properties may be useful, depending on the configuration of the sensor. In one embodiment, the sensor device is first measured by varying a gate voltage applied by a conducting plane beneath the insulator of the substrate. Then the network is exposed to a solution containing sample ssDNA for the period of time disclosed above. Next, the solution is removed, and a period of time is allowed to lapse sufficient for the substrate to become substantially dry. This period of time may be made briefer by taking actions which speed the drying process. For example, dry air may be blown over the substrate. After the substrate is dry, the sensor device is measured again by varying the gate voltage. The resulting measurement is compared to the first measurement to see if dsDNA is present.

In another embodiment, the network is exposed to pure water. The sensor device is first measured by varying a gate voltage applied by a conducting plane beneath the insulator of the substrate. Then the network is exposed to a solution of sample DNA in pure water. While the network is exposed, the sensor device is measured by varying the gate voltage. If the sample DNA contains target DNA, hybridization occurs over time, and the resulting measurement of the sensor device changes in comparison to the first measurement.

In another embodiment, the network is exposed to a conducting liquid. Preferably, the conducting liquid is a buffer appropriate for physiological fluids; most preferably, the conducting liquid is phosphate buffer solution (PBS). The sensor device is first measured by varying a gate voltage applied by a conducting element in contact with the conducting liquid. Then the network is exposed to a solution of sample DNA in a similar conducting liquid. While the network is exposed, the sensor device is measured by varying the gate voltage. If the sample DNA contains target DNA, hybridization occurs over time, and the resulting measurement of the sensor device changes in comparison to the first measurement.

At step 540, the observed electrical response should be correlated to the target species to determine a positive or negative result. For example, with gene testing, the target sequence is ether present, or it is not. Reaction between the sensor and the targeted gene sequence should produce results that are consistent and repeatable for sensors of a given type. Thus, a positive or negative result, and a confidence level, may be based on a comparison between a particular sensor response and statistical control data for sensors of the same type. Confidence in a result may be increased by performing multiple measurements using multiple sensors in parallel.

### EXAMPLE A

A degenerately doped silicon wafer with a silicon oxide film was coated with carbon nanotubes in a random network, as described in the earlier-referenced U.S. patent application Ser. No. 10/177,929 and generally in accordance with the description hereinabove. Titanium contacts 30 nm thick covered with gold contacts 120 nm thick were deposited and patterned by photolithography and lift-off to form opposing contacts. The contacts each comprised a plurality of interdigitated portions disposed over a generally rectangular region. A network of randomly oriented nanotubes was disposed over the silicon substrate. Nanotubes in the network were in electrical contact with interdigitated portions of the contacts. After the deposition of the contacts, nanotubes outside of the generally rectangular area were removed by oxygen plasma etching, leaving nanotube network remaining. The use of interdigitated sets of metal electrodes with nanotube network interposed generally between the interdigitated contacts results in many nanotubes connected in parallel across the electrodes.

A die was separated from the wafer and mounted in a standard 40-pin chip carrier, with wires connecting the interdigitated wires on the chip to the contacts on the chip carrier. The contact pads and wires on the packages were coated with epoxy resin, which was allowed to cure. Chips in packages thus prepared were rinsed with acetone, isopropanol, deionized water, and then 18 MΩ water.

A solution of oligonucleotide 5′-CCT MT MC MT-3′ at concentration 10-4 M was prepared by dissolving 84500 pmole of the oligonucleotide in 845 μL of pure water (18 MΩ water from a NANOpure Infinity UV water system). A chip prepared as described above was measured by varying a gate voltage applied by a conducting plane underneath the insulator. The resulting curve is shown in FIG. 6 as item 600. Then a drop containing 20 μL of DNA solution was placed on the chip. The chip and solution were placed in a humidified chamber at room temperature for 12 hours. Then the chip was removed from the chamber and rinsed with 18 MΩ water and blown dry with dry nitrogen. The chip was measured by varying the gate voltage. The resulting curve is shown in FIG. 6 as item 610. This curve represents a sensor device prepared for use as a sensor. At this stage, the nanotube network is contacted by ssDNA with a probe sequence. The effect of the ssDNA coating on the electronic measurement is that the curve 610 is shifted to the left of curve 600.

To demonstrate contact between the nanotubes and the probe ssDNA, one chip was prepared with a labeled ssDNA. Labeled ssDNA is not necessary for the preferred embodiment and is only described here for illustrative purposes. A solution of oligonucleotide 5′-HS-(CH2)6-CCT MT MC MT-fluorescein-3′ at concentration 10-5 M in 18 MΩ water was prepared as a receptor DNA sequence. A chip was exposed to this solution overnight, rinsed, and dried with nitrogen gas. An optical fluorescence micrograph of this chip was observed, and a green fluorescein label appeared as a bright area only in a defined area where the nanotube network was present, and not in other areas of the substrate. This demonstrated that the receptor DNA strand was attached to the nanotubes of the sensor.

Next, a solution of target DNA, oligonucleotide 5′-ATT GTT ATT AGG-3 complementary to the receptor DNA strand, at concentration 10-4 M, was prepared by dissolving 132,000 pmole of the oligonucleotide in 1320 μL of 18 MΩ water. A diluted solution of target DNA at concentration 10-8 M was thereby prepared. The chip was exposed to a 20 μL drop of this solution in a humidified chamber at room temperature for one hour. The chip was then removed from the chamber and rinsed with 18 MΩ water and blown dry with dry nitrogen.

FIG. 6 shows the resulting curve as item 620. This curve represents the result of hybridization of the probe DNA with the target DNA. The effect of the target DNA hybridization on the electronic measurement is that the curve 620 is shifted to the right of curve 600.

### EXAMPLE B

### Noncovalent Chemical Functionalization of Carbon Nanotube Devices for Single Base Mismatch DNA detection.

B-1. Summary:

In one exemplary embodiment having aspects of the invention, a nanotube sensor device comprises a carbon nanotube network field effect transistor (“NTFET” or “NTNFET”) device functionalized with single-stranded DNA (ssDNA). In certain embodiments, single-stranded DNA (ssDNA) may be immobilized on NTFET devices through polymer and polyaromatic molecules non-covalently attached to carbon nanotubes. The significant differences in the electronic response of functionalized NTFETs to complementary single-stranded DNA (cDNA) and single base mismatch single-stranded DNA (sbmDNA) may be measured. This exemplary sensor includes the following structure, elements and functions:

a) One or more carbon nanotube FET device comprising a single nanotube and/or a networks of nanotubes disposed to form a conducting channel between at least a source and a drain electrode.

b) The FET geometry may include a bottom gate electrode and/or a liquid gate electrode.

c) Polymer and/or aromatic linker molecules attached non-covalently to the carbon nanotubes

d) ssDNA is attached chemically to the linker molecule to create a probe.

e) In operation, when complementary cDNA is exposed to the sensor, it hybridizes with the probe, with a measurable effect on device electrical characteristics.

In operation, when single base mismatch sbmDNA is exposed to the sensor, it also hybridizes with the probe, but produces measurably distinct device characteristics.

NTNFET devices were prepared according to procedures further described, among other places, in U.S. patent application Ser. Nos. 10/177,929, 10/656,898, and 10/704,066, each incorporated by reference above. Electric current is an electrical property that may be measured using contacts. A contact comprises a conducting element that may be disposed on the substrate, such that the conducting element is in electrical communication with the nanotube network. At least two contacts may be placed within the defined area of the nanotube network, such that each contact is in electrical communication with the network.

In some embodiments of the invention, an additional conducting element, referred to as a gate electrode, is provided such that it is not in electrical communication with the at least one nanotube, but such that there is an electrical capacitance between the gate electrode and the at least one nanotube. In one exemplary preferred embodiment, the gate electrode is a conducting plane within the substrate beneath the silicon oxide. Examples of such nanotube electronic devices are provided, among other places, in the above incorporated patent applications Ser. Nos. 10/656,898 and 10/704,066.

The sensor NT devices may be made using standard photolithography techniques on, for example, 100 mm wafers. NTFET devices were fabricated using SWNTs grown by chemical vapor deposition (CVD) at 900° C. using dispersed iron nanoparticles as growth promoter and a methane/hydrogen gas mixture. Electrical leads were patterned on top of the nanotubes from titanium films 30 nm thick capped with a gold layer 120 nm thick. After conducting initial electrical measurements to establish the device characteristic, the substrates were wire bonded and packaged in a 40-pin CERDIP package before conducting the DNA experiments. The contact pads and wires on the packages were coated with epoxy resin, which was allowed to cure. The DNA experiments were performed by putting a single drop of the DNA solution on the package, which is located in a sealed jar, containing a beaker with ˜100 mL of water to prevent the evaporation of the drop.

Electronic measurements of NTFET devices, such as current flow between S/D electrodes as a function of applied gate voltage, were conducted using Parallel Measurement System (PMS). This system is capable of measuring device characteristics of up to 12 nanotube-based sensors simultaneously. A set of 32 independent analog switches are digitally controlled via PC and allow the user to select the junctions to be measured. Applied source-drain bias and gate voltage are both user defined (amplitude, frequency, function). The system can measure device conductance as both a function of time and gate-voltage.

B-2 Preparation Procedures:

B-2.1 Preparation of Chips. Before each chip was used, it was packaged and the wires and contacts were coated with epoxy, which was allowed to cure. The chip was rinsed from a squirt bottle with acetone, isopropanol, deionized water, and finally was washed using the formalized washing procedure (Section B-2.2), after which initial I-Vg curves were taken.

B-2.2 Washing Procedure. A packaged chip was briefly rinsed with a squirt of 18 MW water to remove any analyte on the surface. In a crystallizing dish, approximately 50 ml of a 0.01 M Phosphate buffered saline solution (pH 7.4 @25° C.) was poured over the chip. It was washed on an orbital shaker at speed setting 6 for 5 minutes. The solution was then discarded. The chip was then washed four times with 18 MW water in the same way.

B-2.3. I-Vg Curves. While I-Vg curves (plots of NTFET current versus scanned gate voltage) were captured for all devices on the chip, only one for each chip is shown in this report. The curve that is shown in each case should be considered to be representative of all curves obtained for each chip.

B-3 Pyrene-labeling and DNA:

B-3.1. Formation of Pyrene Monolayer. A packaged chip (in this case, W517 26:21) was cleaned and initial I-Vg measurements were taken. A 2.5 mg/mL solution of pyrene butanoic acid succinimidyl ester in N,N-dimethylformamide (DMF) was prepared by dissolving 3.08 mg of the pyrene substance in 1.232 mL of DMF. 50 mL of this solution was placed on the surface of the chip, which was then sealed inside of a chamber for 2 hours at room temperature with an open container of DMF to prevent the drop from evaporating. The chip was then removed, was rinsed with DMF, acetone, and isopropanol, and was then cleaned (Section 2.2), and I-Vg curves were taken.

B-3.2 Covalent Attachment of DNA. 20 mL of the DNA-NH2 solution was placed on the surface of the chip, which was then sealed inside of a chamber overnight at room temperature with an open container of water to provide humidity and prevent the drop from evaporating. The chip was then removed, was washed according to the washing procedure, and I-Vg curves were taken.

B-4 Detecting DNA Hybridization:

B-4.1 Detection Procedures. A 10-6 M solution of the DNA oligonucleotide was prepared by diluting 10 mL of a 10-4 solution of the oligonucleotide with 990 mL of a 0.01M Phosphate buffered saline solution (pH 7.4 @25° C.). 20 mL of this DNA solution was placed on the surface of a chip, which had been functionalized with a DNA-pyrene layer according to Section B-3. The chip was then sealed inside of a chamber overnight at room temperature with an open container of water to provide humidity and prevent the drop from evaporating. The chip was then removed, was washed, and l-Vg curves were taken.

B 4.2 cDNA. A chip (W517 26:21) was functionalized according to Section B-3, and was then treated with cDNA according to Section B4.1.

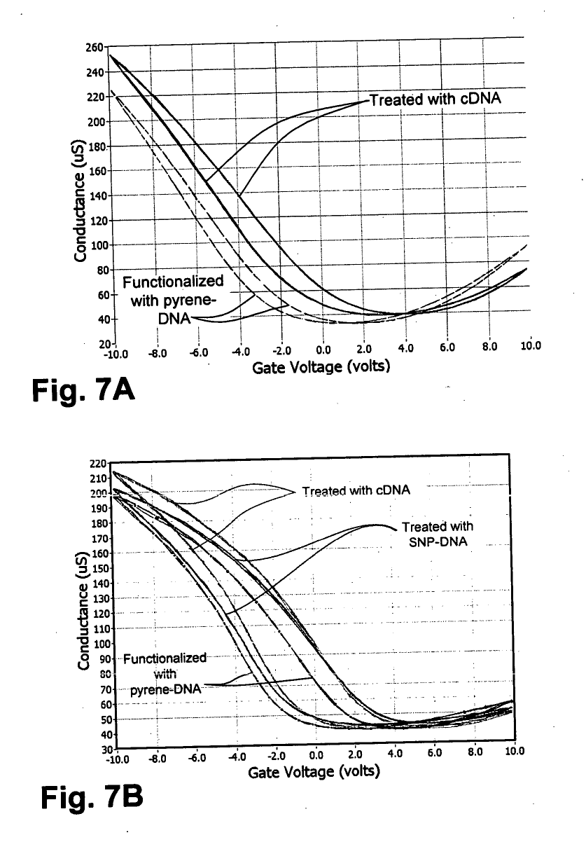

FIG. 7A shows the l-Vg curve, which reveals that the curve is shifted to the right, suggesting that the device can detect the hybridization of the covalently bound DNA with the cDNA. A shift to the right is consistent with shifts seen in previous experiments when double stranded DNA is present.

B4.3. SNP-DNA. A chip (W517 26:24) was functionalized according to Section B-3, and was then treated with SNP-DNA according to Section B4.1.

FIG. 7B shows the I-Vg curve, which reveals a fairly insignificant shift to the right. This may be due to partial (but incomplete) binding of the SNP-DNA to the DNA attached to the device, or it may be that the SNP-DNA is washed away during the washing procedure, as this magnitude of shift has also been shown to be associated with drift (possibly due to a very thin layer of water adsorbed to the nanotubes). Either way, it can be asserted that the devices are able to distinguish between cDNA and a SNP.

B4.4. SNP-DNA+cDNA. The chip (W517 26:24) that had already been treated with SNP-DNA was then treated with cDNA according to Section B-4.1.

FIG. 7B shows the l-Vg curve and reveals a shift to the right, which is similar to the shift seen with cDNA in Section B-4.2. This indicates that the device can detect the cDNA after being exposed to the SNP-DNA. If the SNP-DNA was not washed away in Section B-4.3, then the cDNA can displace the SNP-DNA, producing a result that is consistent with the data seen for hybridization in Section B-4.2 and elsewhere.

The nanoscale electronic devices, NTFETs, may be used for real time monitoring and detection of nucleic acids (RNA and DNA) in small quantities. For DNA oligonucleotide hybridization assays, the NTNFET devices can detect a small amount of single-stranded DNA (ssDNA). Such assays are faster and much more sensitive than existing methods and, for example, reduce the necessary number of DNA duplication cycles or even eliminate PCR.

When there is a single mismatched base between two DNA strands, hybridization can still occur but the hybridization complex with the “kink” due to the mismatch will be less stable. These mismatches are called single nucleotide polymorphism (SNP), and were discovered as a result of the Human Genome Project. SNPs are the key target for commercial genetic tests and can be potentially identified by NTNFET devices.

### EXAMPLE C

DNA assays using nanoelectronic devices.

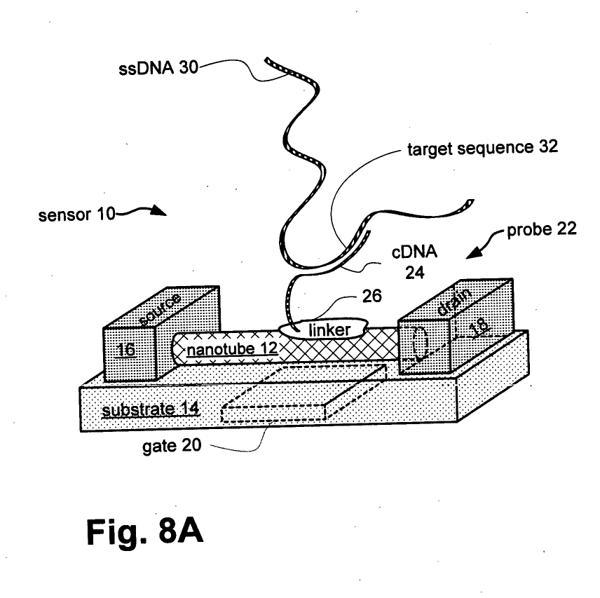

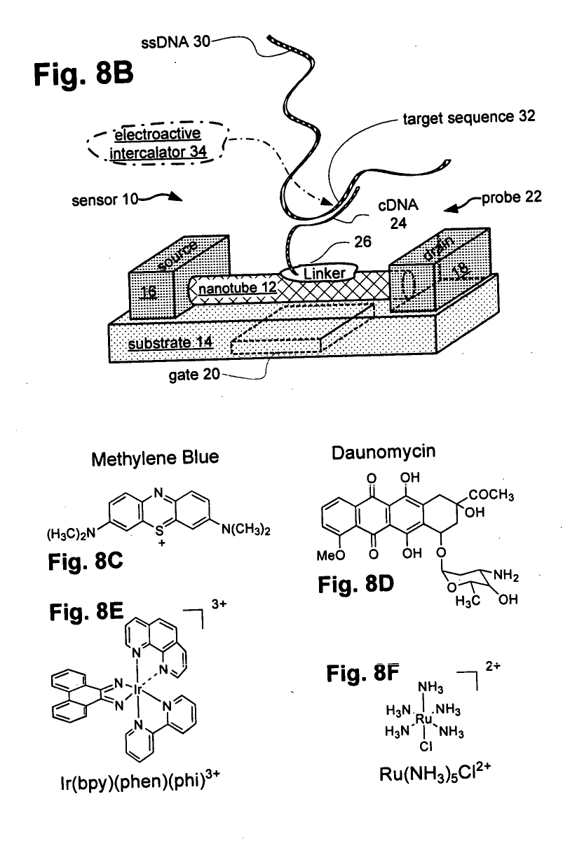

A number of different exemplary DNA (or other polynucleotide) assay embodiments having aspects of the invention are shown in FIGS. 8-12. The structure and methods shown are exemplary, and other alternative embodiments may use structures and methods described elsewhere in this application. Where the different embodiments include substantially similar elements, the same reference numbers are used to designate such elements in the description of each embodiment.

C-1 Structure:

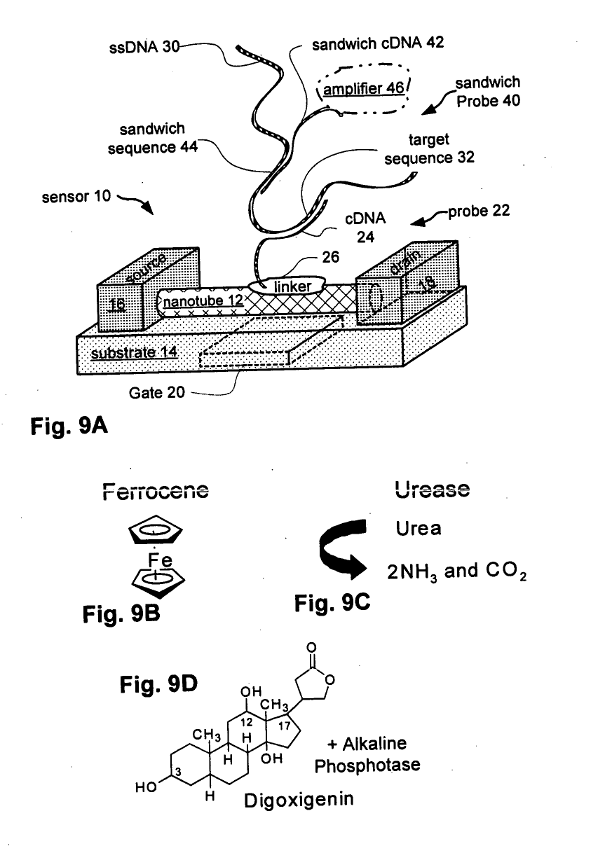

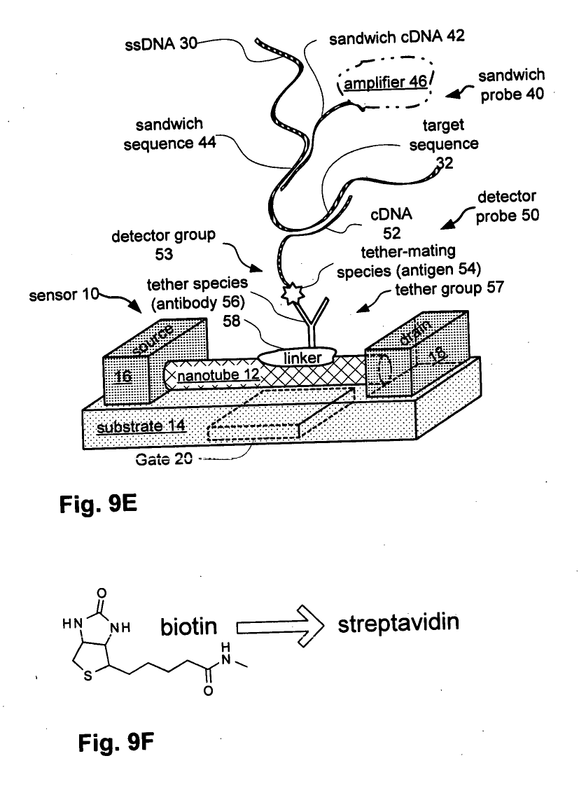

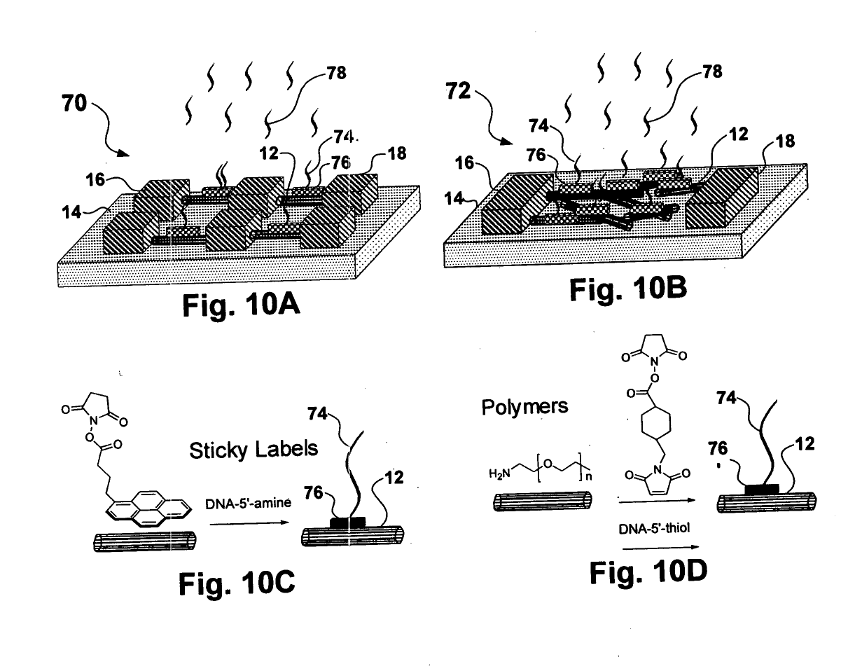

As shown in FIGS. 8-9, and also in FIGS. 10-12, the sensor 10 comprises a platform having at least one nanostructure, such as nanotube 12 disposed adjacent substrate 14 and in electrical communication between at least a source electrode 16 and a drain electrode 18.

Optionally, the device may include at least one additional electrode, such as gate electrode 20 disposed adjacent nanotube 12. The gate electrode 20 is shown embedded in substrate 14, but alternative electrodes types and locations may be included (e.g., a bottom gate electrode, top gate and/or liquid gate electrode), as described above with respect to other NTFET sensor embodiments.

Although a single nanotube 12 is shown schematically in FIGS. 6-9 in simple end-contact with electrodes 16 and 18, alternative nanostructures arrangements may be employed as described above without departing from the spirit of the invention.

FIGS. 10A and B show schematically two alternative configurations. FIG. 10A shows a plurality of conductor “islands” interconnected by nanotubes, and FIG. 10B shows a nanotube network embodiment, in which plurality of nanotubes form an interconnecting network or film of nanotubes providing a conducting channel between source and drain electrodes. In such a nanotube film, individual nanotubes need not span between source and drain electrodes, and the conducting channel may comprise one or more channels or paths via a plurality of nanotubes connected to one another in series. Preferably, the density and/or composition of such a network of nanotubes is selected (by controlled formation and/or by post-formation modification) to provide a desired degree of conductivity and sensor sensitivity. Optionally, a plurality of source and/or a plurality of drain electrodes may be included, for example an interdigitating series of such electrodes. The nanotube 12 may be disposed to lie under, beside or above the electrodes, or combinations thereof.

Nanotube films may be made directly on the substrate, e.g. by nanodispersed-catalyst-mediated CVD, solution deposition and the like. Alternatively, a nanotube film may be made separately and deposited upon the substrate 14 as a separate step, either directly or including a film carrier layer. See patent application Ser. Nos. 10/177,929 and 10/846,072 incorporated above. Note that substrate 14 may be a rigid structure, e.g. a semiconductor wafer, monocrystalline silicon, polycrystalline silicon, or the like, or alternatively may be flexible, e.g. a polymer sheet, web, or the like. Portions of the nanotube film may be selectively removed from portions of the substrate so as to tailor the nanotube film in relation to the electrodes 16 and 18. Likewise, the contacts or electrodes 16 and 18 (and/or gate or additional electrodes) may be deposited or formed prior to the nanotubes 12 or afterwards. Optionally, additional electronic circuitry may be formed integrally with sensor 10 on substrate 14, e.g. for signal processing and the like. Known methods for constructing elements and layers of integrated electronic circuitry may be employed in the making of sensor 10 and optional elements, such as CVD, vacuum deposition, photolithography and masking, chemical etching, spin coating, substrate doping, substrate oxide formation, substrate nitride formation and the like.

Likewise, the sensor shown may be included in an integrated array of sensors, as described above. Note that the enhancements and alternative elements describe above with respect to other sensor and NTFET embodiments, such as passivation of contacts, dielectric and/or catalyst containment layers covering the substrate, hydrophobic coatings on the nanostructures, and the like, may optionally be included in the embodiments described below.

C-2 Detecting probes:

As shown in FIGS. 8-9 and FIG. 10, the sensor includes a detecting probe, such as probe 22, the probe includes a linker group, such as linker 26 which is associated (preferably non-covalently) with the nanotube 12, so as to bind the probe to the sensor 10. A cDNA 24 is bound to linker 26 (preferably covalently) at one portion of the cDNA, the cDNA also having an exposed complement base sequence extending outward from linker 26. The linker may be a molecule or group configured to non-covalently bind to nanotube 12 and to covalently bond to cDNA 24, e.g., an aromatic molecule such as pyrene and/or a polymer.

Note that a linker group may connect to more than one cDNA, and conversely a cDNA may connect to more than one linker group, depending on the nature and conformation of the linker. For example a liner group comprising a distributed polymer layer may have a plurality of cDNA molecules bonded at different points on the polymer layer.

Note in this regard the definitions of dsDNA, ssDNA, cDNA and other nucleotide species set forth in the Summary of the Invention herein.

Thus, in certain alternative embodiments, the “cDNA” is not necessarily a deoxyribose polynucleotide, but may include other target-specific polynucleotide species, such as RNA, a modified or substituted DNA, and the like, having a detector nucleotide sequence which provides for at least partial hybridization with a selected target sequence.

Similarly, the target “ssDNA” molecule is not necessarily a discrete fully-denatured deoxyribose polynucleotide strand, but may include RNA, dsDNA, partially-denatured dsDNA, species with “sticky ends”, and the like, wherein the target molecule includes a target nucleotide sequence which provides for at least a partial hybridization with the “cDNA” of the probe.

In the embodiment shown in FIG. 8A, the probe 22 is shown detecting a single-stranded fragment of DNA 30 by hybridizing with target base sequence 32. Suitable sensor circuitry (not shown in FIGS. 8-12) is connected to sensor 10 so as to detect and/or quantify an electrical response of sensor 10 to the hybridization of DNA 30, in a manner described above with respect to other sensor embodiments. For example, the conductance between source 16 and drain 18 may change upon hybridization, the change being measured. Alternatively, in an NTFET DNA sensor embodiment, the hybridization of DNA 30 may cause a phase shift in the device characteristics of sensor 10 produced as the voltage of gate electrode 20 is varied through a selected voltage range. Additional or alternative properties of sensor 10 may be measured to detect hybridization.

In certain applications according to aspects of the invention, the sensor 10 may be used to discriminate between a relatively complete hybrid match between cDNA 24 and selected target sequence 32 on the one hand, and a contrasting partial, discontinuous, and/or or looped hybridization of the target sequence on the other hand. The sensor 10 produces an electrical response to the hybridization event with signal characteristics reflecting the degree and/or character of hybridization of probe cDNA 24 to a target sequence 32. For example, the signal produced upon partial hybridization of a sequence which has a single base mismatch (sbmDNA) relative to the corresponding probe sequence can be distinguished from the hybridization of a completely matched sequence. This capability of sensor 10 provides for the characterization of single nucleotide polymorphisms (SNPs), among other things.

Note in this regard the ssDNA 30 may alternatively be a RNA polynucleotide, a hetero or modified polynucleotide, a plasmid, a viral fragment, a double stranded DNA fragment (e.g. having a “sticky end” or other exposed strand target portion available for hybridization with probe 22), a partially-annealed dsDNA fragment, an oligonucleotide, or the like.

In a exemplary method of use according to aspects of the invention, the probe 24 may be prepared to suit a selected target sequence 32, the cDNA being obtained by known methods. Commercial sources exist for custom, synthesis of oligonucleotides having a specified sequence, and sequences of interest may also be obtained, modified and/or amplified by a number of known methods, such as PCR, reverse transcription, plasmid amplification, and the like. Note in this regard that cDNA may contain nucleotides and/or hetero-groups in addition to a nucleotide sequence complementary to target sequence 32, for example, tail or head portions selected for binding to linker 26, selected for purification, amplification and/or other processing steps, optional labeling groups, and the like. The cDNA 24 may then be bonded to linker group 26 (e.g. pyrene) by known reactions and methods (e.g., formation of a DNA-5′-amine of pyrene) to create probe 22.

Prefabricated sensor platforms 10 may then be functionalized, for example by treatment with a solution or suspension of probe 22 so as to bind linker 26 to nanotube 12 (e.g., by pi-pi stacking of pyrene molecules associated with the graphitic lattice of nanotube 12), followed by washing and drying. The functionalized sensor 10 may then be used for detection of an analyte ssDNA having target sequence 32, suspended in a sample medium. Suitable calibration procedures may be carried out, e.g. by exposing sensor 10 to an equivalent sample medium having ssDNA known to lack target sequence 32.

In an alternative exemplary method of use, the prefabricated sensor platforms 10 may be pre-treated with a linker group material 26 (e.g., a polymer selected to react with or bind to a portion of cDNA 24). A target-specific cDNA 24 may be prepared, and the sensor 10 functionalized by binding with the cDNA 24 to create probe 22 in situ.

In an alternative sensor embodiment (not shown) according to aspects of the invention, an array sensor system comprises a space-apart plurality of individual sensors 10. The array may be prefabricated as described above, and the sensors 10 may be individually functionalized with one of a plurality of different probes, each having cDNA specific to a particular selected target sequence. For example, ink-jet type application methods may be used to treat the array in a predetermined pattern of functionality. Such a multi-functionality array may be employed so that a single analyte sample medium may be tested for a plurality of different target DNA sequences substantially simultaneously. Signal processing circuitry of known design may be used to process signals from the plurality of sensors 10 of the array serially, in parallel, or according to any selected pattern. Accessory elements, such as microfluidic reservoirs, channels, needle, valves, pumps, and/or injectors, and the like, may be included in the array embodiment, configured to provide controlled functionalization of the sensors, controlled sample delivery to the sensors, sample purging from the sensors, washing/reconditioning of the sensors, and/or controlled calibration of the sensors, and the like.

C-3 Alternative Assay Embodiment

FIGS. 8B and 9 illustrate a number of alternative assay embodiments according to the invention, one or more of which may be employed instead of or in combination with the embodiments described above.

FIG. 8B shows schematically an alternative exemplary embodiment according to aspects of the invention, employing an electroactive intercalator 34, either in the sample medium and/separately introduced following hybridization. The intercalator 34 associates with the hybridized portion (double stranded region) of the probe 22-target sequence 32 complex, so as to amplify and/or modify the measured response of sensor 10, so as to facilitate measurement and/or detection of hybridization.

As shown in the structures of FIGS. 8C-F, examples include the use of electroactive intercalators such as daunomycin, methylene blue, Ir(bpy)(phen)(phi)3+, and the like; groove binders, such as Ru(NH3)5Cl2+, and the like; or combinations thereof.

FIG. 9A shows schematically an alternative exemplary assay embodiment according to aspects of the invention, employing an secondary or sandwich probe 40, configured to hybridize with a second portion of ssDNA 30, referred to as “sandwich sequence” 44. The sandwich probe 40 includes a second cDNA 42 having a portion including a sequence of bases complementary to sandwich sequence 44. The cDNA 42 includes a portion which is in turn bound to an amplifier group 46, preferably covalently. The amplifier group 46 serves to increase or modify the signal response of sensor 10 upon hybridization of target sequence 32 to detector probe 22. The amplifier group 46 may be a group or label which causes a detectable and/or a quantifiable signal of sensor 10 without further reactivity. Alternatively, amplifier group 46 may be a group which causes a detectable and/or a quantifiable signal of sensor 10 upon further reaction with another promoter material, such as a chemical or biochemical substrate. Examples of amplifier groups are shown in FIGS. 9A-C.

There are a number of alternative methods of use embodiments according to aspects of the invention for the assay shown in FIG. 9A. For example, comprising:

a) bonding probe 22 to nanotube 12 of sensor 10;

b) treating sensor 10 with a sample putatively containing analyte ssDNA 30 having target sequence 32, so as to bind ssDNA 30, if present, to probe 22, followed by washing;

c) treating sensor 10 with a solution containing sandwich probe 40 having selected amplifier group 46, so as to bind probe 40 to ssDNA 30, if present, followed by washing;

d) if needed for the selected amplifier 46, treating sensor with a solution including the further promoter material;

e) acquiring a signal from sensor 10; and

f) analyzing the signal to determine the presence and/or concentration of analyte ssDNA 30.

The steps a-f above may be carried out in alternative order. For example, step (c) may be a pretreatment of treatment of the analyte sample, carried out prior to step (b). Likewise, additional calibration steps may be optionally included at various times. The washing steps are exemplary, as one of ordinary skill in the art will readily be able to tune or optimize the methods embodiments for particular applications to avoid cross contamination and other sources of error, without undue experimentation and without departing from the spirit of the invention.

Note that in certain embodiments, the sandwich sequence 44 may be a common sequence expected to be present in the sample DNA fragments, and target sequence 32 may be an analyte-specific sequence of unknown presence in the sample. Alternatively, probe 46 may be configured to undergo relatively non-specific binding to sample DNA in comparison to more highly target-specific binding of probe 22. In this regard, probe 46 optionally may include additional groups to promote binding to sample DNA and/or to prevent undesired blocking of probe 22.

In certain embodiments, amplifier group 46 may be comprise a promoter or catalyst, such as an enzyme, causing an oxidation/reduction or other reaction with a chemical or biochemical substrate thereby influencing sensor 10 to provide a detectable response. For example, amplifier group 46 may comprise urease. Step (d) above may comprise treating with a urea solution to produce ammonia and carbon dioxide if bound probe 46 is present, so as to modify the pH of the solution and thereby detectably change the signal of sensor 10. Other examples of enzyme systems which may be employed are cholinesterase; peroxidase (e.g. HRP); glucose oxidase, and the like. Other examples of amplifier group 46 are ferrocene, metal nanoparticles, labels (nanoparticles, proteins, etc.), and the like.

FIG. 9E shows schematically an alternative exemplary assay embodiment according to aspects of the invention. In this alternative assay embodiment, the sandwich probe 40 and ssDNA 30 are generally similar to that shown and described with respect to FIG. 9A.

However, in the embodiments illustrated in FIG. 9E the detector probe 50 comprises a tether group 57 and a corresponding detector group 53, joined or mated to one another. Tether group 57 includes linker 58 connected to a tether species (in this case antibody 56). Detector group 53 includes cDNA 52 connected to a tether-mating species (in this case antigen 54 where the antigen is selected to have epitopes configured to bind to the receptors or binding sites of antibody 56).

For example, as shown in FIG. 9E, the antigen 54 may comprise biotin and the antibody 56 may comprise streptavidin. In other alternative embodiments (not shown) the arrangement may be the reverse of that shown in FIG. 9E, e.g., an antigen may be connected to the linker, and the antibody connected to the cDNA. Other alternative combinations of tether and tether-mating species may be employed, where the tether and tether-mating species are selected to be readily joinable or mate-able to one another to form the self-assembled detector probe 50.

Note that the tether group 57 and the detector group 53 may generally be prepared separately. For example, a partially-functionalized sensor platform including the tether group 57 may be prepared, and provided and stored without the detector group 53. Such a sub-assembly does not have vulnerability to substances or conditions that may specifically degrade polynucleotides (for example endonucleases, exonucleases and the like). Thus, this embodiment is especially suitable for applications in which it is desired to prefabricate a sensor assembly without a target-specific cDNA (i.e., a relatively generic sensor), and then introduce any one of a number of different target-specific detector groups which are conveniently joinable or mate-able to the tether group (self-assembly or simple reaction) at the time of, or shortly before, sample measurement. The rapid, robust, and selective antigen-antibody binding reaction is a preferred embodiment of the tether/tether-matching system.

The tether/tether-matching system illustrated in FIG. 9E may also be employed in conjunction with other embodiments having aspects of the invention, such as the exemplary embodiments shown in FIGS. 8-9D, providing similar advantages. Likewise, a particular sensor 10 may be functionalized employing more than one combination of tether/tether-matching system, wherein the detector groups each have the same selected cDNA type (in certain applications where a particular cross-reactivity is desired, more than one kind of cDNA may be employed in a particular sensor. However, more typically, the sensor will be configured to maximize target selectivity)

Additionally, the self assembling tether/tether-mating system is particularly useful in sensor array embodiments having aspects of the invention, in that a relatively generic sensor array can be pre-fabricated with tether groups bonded to the plurality of sensors. A selected pattern of different target-specific detector groups may then be applied by known methods to complete the patterned target-specific functionalization of the sensor array, e.g. by multiple or automated pipette systems or by “ink jet” methodology.

C-4 Alternative Sensor Arrangements:

FIGS. 10A and 10B shows an “island” sensor embodiment 70 and a “nanotube network” sensor embodiment 72, respectively, each according to certain aspects of the invention. These embodiments are generally applicable to the assay embodiments shown in FIGS. 8-9. Nanostructures 12 (in this case, single wall carbon nanotubes, “SWCNT” or abbreviated “NT”) communicate electrically with source electrode 16 and drain electrode 18. In the embodiment 72, a plurality of nanotubes 12 form an interconnecting network between source 16 and drain 18. Linker groups 76 may be seen to connect the cDNA strands 74 to nanotubes 12. The ssDNA strands 78 may be seen to be diffusing in the vicinity of cDNA strands 74.

FIGS. 10C and 10D schematically show the connection of cDNA strands 74 with nanotubes 12 be action of linker groups 76, as exemplified by (FIG. 10C) an organic group 76 (e.g., pyrene) covalently bonded to the cDNA 74 or (FIG. 10D) a reactive polymer group 76′ covalently bonded to cDNA 74, or the like or combinations thereof.

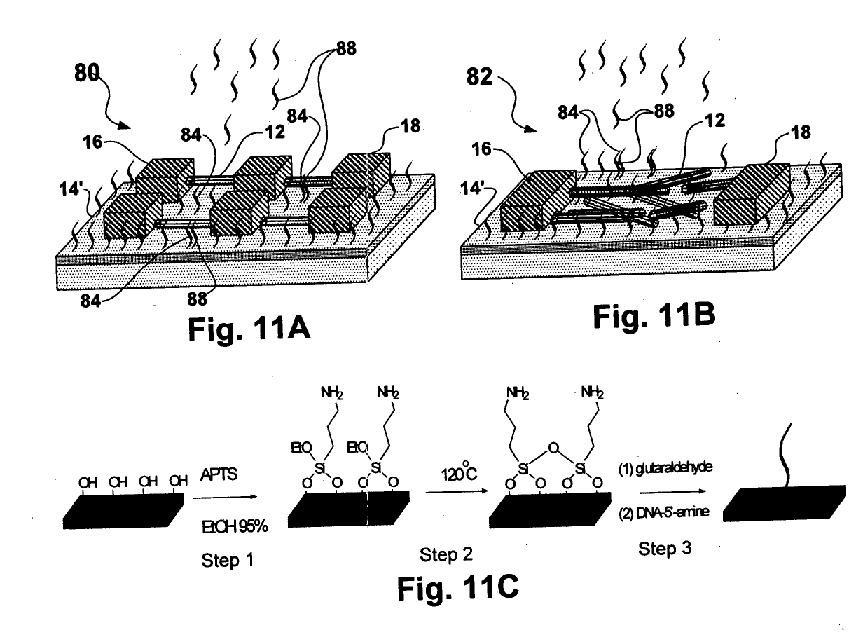

FIGS. 11A and 11B show alternative sensor architectures 80 and 82 in which the cDNA 84 is attached to the surface of substrate 14′ (e.g., a silicon dioxide layer covering a silicon wafer) by means of a chemical connection, such as a covalent bond. FIG. 11C shows the sequence of steps of an alternative linker method to bind the cDNA 84 to the substrate 14, employing known reactants and methods, as shown in the sequence of steps (steps 1-3). Hybridization of ssDNA 88 to cDMA 84 influences the electrical properties of sensor 80 or 82 respectively so as to produce a detection signal generally similar to that described above for the various assay embodiments.

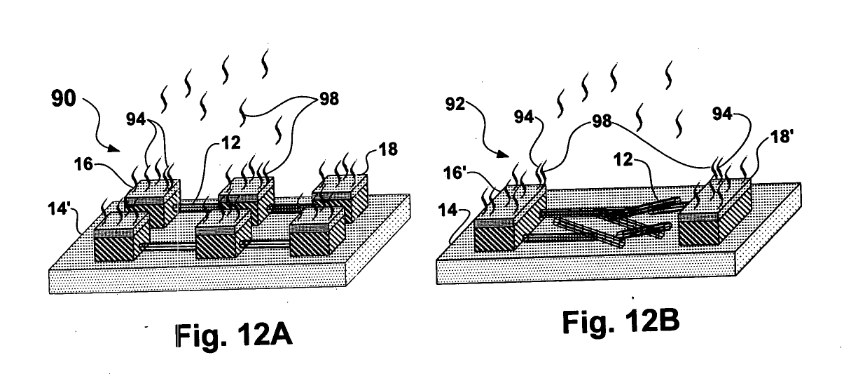

FIGS. 12A and 12B show alternative sensor architectures 90 and 92 in which the cDNA 94 is attached to the surface of electrodes or contacts 16′ and 18′ (electrodes may be bare, oxidized surface, or a coated surface) by means of a chemical connection, such as a covalent bond, e.g., by formation of DNA-5′-thiol at the cDNA 5′ end, employing known reactants and methods. Alternatively, the cDNA 94 may be attached to contacts 16′ or 18′ by polymer linker groups, and the like. Hybridization of ssDNA 98 to cDMA 94 influences the electrical properties of sensor 90 or 92 respectively so as to produce a detection signal generally similar to that described above for the various assay embodiments.

C-5 Alternative Separation and Purification:

Various label groups known in the art may be employed for separation of the target DNA from genomic DNA in the vicinity of the nanotube device 10. For example, labels (nanoparticles, proteins, etc.) may be used for separation of the target DNA from genomic DNA as an additional step. Alternatively magnetic beads and antibodies may be employed for such separators. In certain exemplary embodiments according to aspects of the invention, pre-measurement sample DNA purification and/or segregation, and the like are carried out adjacent the sensor (or adjacent the sensor array in array embodiments) as part of an integrated sample processing and measurement system, and may include magnetic controls, electrostatic controls, combinations of these, and the like. Optionally, a microprocessor or computer element is included to control and coordinate both sample DNA purification and/or segregation and sample detection and measurement.

Having thus described a preferred embodiment of the nanotube sensor device, it should be apparent to those skilled in the art that certain advantages of the within system have been achieved. It should also be appreciated that various modifications, adaptations, and alternative embodiments thereof may be made within the scope and spirit of the present invention. The invention is further defined by the following claims.

What is claimed is:

1. A nanotube sensor for sensing an polynucleotide, comprising:
a substrate;
a first nanotube over the substrate;
at least one conducting element in electrical communication with the first nanotube; and
at least one single-strand DNA molecule operatively associated with the first nanotube, the at least one single-strand DNA molecule configured for interacting with a complimentary target DNA strand to alter an electrical property of the nanotube sensor.

2. The nanotube sensor of claim 1, wherein the at least one single-strand DNA is directed attached to the first nanotube.

3. The nanotube sensor of claim 1, wherein the first nanotube is selected from the group consisting of single-walled nanotubes, double-walled nanotubes, multi-walled nanotubes, and onions.

4. The nanotube sensor of claim 1, wherein the first nanotube comprises at least one element selected from the group consisting of carbon, boron, boron nitride, and carbon boron nitride, silicon, germanium, gallium nitride, zinc oxide, indium phosphide, molybdenum disulphide, and silver.

5. The nanotube sensor of claim 1, wherein the first nanotube comprises a single-wall carbon nanotube.

6. The nanotube sensor of claim 1, wherein the at least one conducting element comprises an electrode including at least one material selected from the group consisting of a metal, carbon and conductive polymer.

7. The nanotube sensor of claim 1, wherein the electrical property of the nanotube sensor is a capacitance of at least the first nanotube, and wherein the sensor further comprises a counter electrode configured to permit measurement of the capacitance.

8. The nanotube sensor of claim 1, wherein the at least one conducting element includes at least two conducting elements, and the conducting elements comprise metal electrodes.

9. The nanotube sensor of claim 8, wherein the conducting elements are in direct physical contact with the first nanotube.

10. The nanotube sensor of claim 8, further comprising a gate electrode in proximity to the nanotube.

11. The nanotube sensor of claim 8, further comprising a layer of inhibiting material covering regions of the sensor adjacent to the connections between the conductive elements.

12. The nanotube sensor of claim 8, wherein the nanotube further comprises a two-dimensional nanotube network disposed over the substrate between the two conducting elements.

13. The nanotube sensor of claim 12, wherein the nanotube network comprises a plurality of randomly-oriented carbon nanotubes.

14. The nanotube sensor of claim 12, wherein the two conducting elements comprise a pair of interdigitated electrodes.

15. The nanotube sensor of claim 12, wherein the at least one single-strand DNA further comprises a plurality of identical DNA receptor strands distributed over the two-dimensional nanotube network.

16. The nanotube sensor of claim 15, wherein the plurality of identical DNA receptor strands are directed attached to nanotubes of the two-dimensional nanotube network

17. A method for making a nanoelectronic sensor, comprising:
disposing a film of nanotubes over electrodes on a substrate;
depositing a solution of single-stranded DNA over the substrate, wherein the single-stranded DNA is configured as a complement to a target DNA sequence; and
drying the solution to leave a deposit of the single-stranded DNA over the substrate.

18. The method of claim 17, further comprising removing the deposit of the single-stranded DNA from the substrate except in a region between the electrodes.

19. The method of claim 17, further comprising depositing the electrodes as a conductive deposit over the substrate.

20. The method of claim 19, wherein the depositing the electrodes step further comprises configuring the electrodes to have a plurality of interdigitated fingers.

21. The method of claim 17, further comprising providing a gate electrode configured to operate on a region between the electrodes.

22. The method of claim 21, further comprising removing the deposit of the single-stranded DNA from the substrate except in the region between the electrodes.

23. The method of claim 17, further comprising coating the electrodes with barrier material prior to the depositing step.

24. The method of claim 17, further comprising preparing the solution of single-stranded DNA comprising an oligonucleotide dissolved in water.

25. A method for sensing a particular target polynucleotide sequence, comprising:
exposing a solution to a nanoelectronic sensor, wherein the nanoelectronic sensor comprises a complementary polynucleotide for the target polynucleotide sequence attached to at least one nanotube in a region between conducting electrodes; and
observing at least one electrical property of the nanoelectronic sensor during the exposing step.

26. The method of claim 25, wherein the exposing step further comprises exposing the solution to the nanoelectronic sensor comprising a field-effect transistor.

27. The method of claim 26, wherein the observing step comprises observing the at least one electrical property comprising a gate voltage.

28. The method of claim 25, further comprising comparing an electrical property observed during the observing step to a corresponding property observed prior to the observing step.

29. An electronic sensor system for target polynucleotides, comprising:
(a) at least one sensor platform having at least one electrical property, the platform including:
a substrate,
at least one electrode disposed adjacent the substrate, and
at least one nanostructured element disposed adjacent the substrate, the nanostructured element in electrical communication with the electrode;
(b) at least one detector probe operatively associated with the sensor platform, the probe including:
a linker group disposed in association with the sensor platform, the linker being connected to one or more of the following: the nanostructured element, the substrate, and the electrode;
a detector biomolecule having a binding affinity to an analyte polynucleotide.
a bonding connection between the linker group and the detector biomolecule; and
(c) electronic measurement circuitry connected to the electrode, and configured to measure the at least one electrical property of the sensor platform.

30. The sensor system of claim 29, wherein detector biomolecule is selected from the group consisting of polynucleotides, transcription factors and transcription promoters.

31. The sensor system of claim 29, wherein the detector biomolecule comprises a detector polynucleotide having at least one nucleotide sequence which is at least partially complementary to a nucleotide target sequence of the analyte polynucleotide.

32. The sensor system of claim 31, wherein the at least one detector probe operatively associated with the sensor platform is configured so as to influence the at least one electrical property upon engagement of the probe with an analyte polynucleotide by at least partial hybridization of the target sequence.

33. The sensor system as in claim 32, wherein
(a) the at least one electrode includes at least two electrodes disposed in a space-apart arrangement adjacent the substrate, the electrodes each in electrical communication with the nanostructured element so that the nanostructured element provides at least one conduction channel between the electrodes, and
(b) the at least two electrodes connected to the electronic measurement circuitry and configured to measure the at least one electrical property of the sensor platform.

34. The sensor system as in claim 32, wherein the nanostructured element includes a network comprising a plurality of interconnecting single-walled carbon nanotubes.

35. The sensor system as in claim 32, further comprising a gate electrode arranged adjacent the nanostructured element and connected to the electronic measurement circuitry, wherein the circuitry is further configured to selectively bias the gate electrode to at least one voltage during measurement of the at least one electrical property of the sensor platform.

36. The sensor system as in claim 32, wherein the bonding connection between the linker group and the detector polynucleotide comprises at least a chemical bond between the linker group and the detector polynucleotide.

37. The sensor system as in claim 36, wherein the bonding connection between the linker group and the detector polynucleotide comprises at least a covalent bond between the linker group and the detector polynucleotide.

38. The sensor system as in claim 37, wherein the linker group includes an aromatic compound configured to interact non-covalently with the nanostructure.

39. The sensor system as in claim 32, wherein the bonding connection between the linker group and the detector polynucleotide molecule comprises:
at least one tether group connected to the linker group;
at least one tether-mating group connected to the detector; and
wherein the tether group and the tether-mating group (collectively tethering species) have a mutual affinity promoting a self-assembled bonding connection between the tether group and the tether-mating group.

40. The sensor system as in claim 39, wherein the at least one of the tethering species comprises a non-biological molecule.

41. The sensor system as in claim 39, wherein the at least one of the tethering species comprises a biopolymer.

42. The sensor system as in claim 39, wherein the at least one of the tethering species comprises a synthetically-produced polymer substantially equivalent to a naturally occurring biopolymer.

43. The sensor system as in claim 39, wherein:
the at least one tether group includes an antibody connected to the linker group;
the at least one tether-mating group includes a corresponding antigen connected to the detector; and
wherein the mutual affinity between the tether group and the tether-mating group includes the affinity of the antibody binding site to an epitope of the antigen.

44. The sensor system as in claim 39, wherein:
the at least one tether group includes an MHC receptor connected to the linker group;
the at least one tether-mating group includes a corresponding binding peptide connected to the detector; and
wherein the mutual affinity between the tether group and the tether-mating group includes the affinity of the MHC receptor for a corresponding peptide.

45. The sensor system as in claim 39, wherein:
the at least one tether group includes a mammalian cell surface receptor connected to the linker group;
the at least one tether-mating group includes a corresponding specific binding ligand of the receptor, the ligand connected to the detector; and
wherein the mutual affinity between the tether group and the tether-mating group includes the affinity of the cell surface receptor for its corresponding ligand.

46. The sensor system as in claim 39, wherein:
the at least one tether group includes a viron host-attachment-promoting surface group and/or a viron endocytosis-promoting surface group connected to the linker group;
the at least one tether-mating group includes a corresponding mammalian cell surface receptor, the receptor connected to the detector; and
wherein the mutual affinity between the tether group and the tether-mating group includes the affinity of the mammalian cell surface receptor for the corresponding viron surface group.

47. The sensor system as in claim 39, wherein the tether group binding species and the tether-mating group mating species are reversed in attachment order in that the at least one tether group ;is connected to the detector, and the at least one tether-mating group is connected to the linker group.

48. The sensor system as in claim 39, wherein one or more of the tethering species is synthetic.

49. The sensor system as in any of claim 39, wherein the system further comprises an plurality of said sensors, and wherein different ones of the plurality of sensors include pairs of tethering species with distinctly different mutual binding affinities, so as to permit self assembly of a selected multiplex pattern of different target-specific cDNA probes with respect to two or more of the plurality of sensors.

50. The sensor system of claim 29, wherein the electrical property of the sensor platform is a capacitance of at least the nanostructured element, and wherein the sensor system further comprises a counter electrode configured to permit measurement of the capacitance.
